(19) United States
(12) Patent Application Publication
(10) Pub. No.: US 2001/0001666 A1
(43) Pub. Date: May 24, 2001

(54) Liquid skin treatment

(76) Inventor: Marie Harbeck, Austin, TX (US)

Correspondence Address: MARIE HARBECK, 3202 CLUMPGRASS COVE, AUSTIN, TX, 78735, US

(21) Appl. No.: 09/753,901

(22) Filed: Jan. 3, 2001

Related U.S. Application Data
(63) Continuation-in-part of application No. 09/248,573, filed on Feb. 11, 1999.

(51) Int. Cl. B65D 5/72; A01N 59/14; B65D 25/40; B65D 35/38; A61K 33/22; A61K 35/78; A01N 65/00

(52) U.S. Cl. 424/401; 424/658; 514/725; 514/863; 514/887; 514/937; 222/575; 424/736; 424/765; 424/775; 424/729; 514/458

(57) ABSTRACT

A composition for the maintenance of smooth, radiant, hydrated skin, including sufficient amounts of acids of hydroxyacid, to facilitate exfoliation of skin in a novel manner, without substantial irritation, as well as the prevention, treatment and management of skin conditions, such as acne, wrinkled, irregularly pigmented, photoaged, and thickened layers of the epidermis, and the like, which has as its constituents, safflower oil, flaxseed oil, sweet almond oil, apricot kernel oil, jojoba oil, organic beeswax, stearic acid, cetearyl alcohol, cocoa butter, vitamin A and tocopheral linoleate, borax, oil of lavender, and tincture of benzoin as an antimicrobial agent.

This invention is a “Continuation-In-Part” application for Ser. No. 09/248,573 filed Feb. 11, 1999, for Marie Harbeck.

### BACKGROUND—FIELD OF INVENTION

This invention relates to an improved and useful transdermal composition for treatment of skin, including a sufficient amount of an acidic component of hydroxyacid, to facilitate exfoliation of skin in a moist, without substantial irritation manner, as well as the prevention, alleviation and management of skin conditions, such as acne, rosacea, wrinkled, irregularly pigmented, aged, rough and thickened layers of the epidermis, and the like, which has as its constituents, safflower oil, flaxseed oil, sweet almond oil, apricot kernel oil, jojoba oil, tocopheral linoleate, organic beeswax, and tincture of benzoin as an antimicrobial agent.

To the best of the applicant's knowledge the following is the most relevant prior art:

| | | | |
|---|---|---|---|
| U.S. Pat. No. | 4,740,367 | Force, et al. | 1988 |
| U.S. Pat. No. | 5,244,679 | Freston. | 1993 |
| U.S. Pat. No. | 5,489,429 | Griat, et al. | 1996 |
| U.S. Pat. No. | 5,683,704 | Ohba, et al. | 1997 |
| U.S. Pat. No. | 4,883,659 | Goodman, et al. | 1989 |
| U.S. Pat. No. | 4,608,392 | Jacquet et al. | 1986 |
| U.S. Pat. No. | 5,431,911 | Reynolds. | 1995 |
| U.S. Pat. No. | 5,874,463 | Ancira . . . | 1999 |
| U.S. Pat. No. | 5,730,991 | Rapaport. | 1998 |
| U.S. Pat. No. | 6,071,541 | Murad. | 2000 |
| U.S. Pat. No. | 6,159,480 | Tseng, et al. | 2000 |
| U.S. Pat. No. | 5,441,740 | Ozlen. | 1995 |
| U.S. Pat. No. | 6,110,966 | Pollock. | 2000 |
| U.S. Pat. No. | 5,652,266 | Bobier-Rival, et al. | 1997 |
| U.S. Pat. No. | 5,730,991 | Rapaport. | 1998 |
| U.S. Pat. No. | 4,105,782 | Yu, et al. | 1978 |
| U.S. Pat. No. | 5,961,999 | Bimczak. | 1999 |
| U.S. Pat. No. | 5,958,984 | Devillez. | 1999 |
| U.S. Pat. No. | 5,863,943 | Groh. | 1999 |
| U.S. Pat. No. | 5,776,917 | Blank, et al. | 1998 |
| U.S. Pat. No. | 6,110,448 | Fuller. | 2000 |
| U.S. Pat. No. | 6,069,169 | Ptchelintsev. | 2000 |
| U.S. Pat. No. | 6,017,548 | Epstein, et al. | 2000 |
| U.S. Pat. No. | 5,993,832 | Lorant, et al. | 2000 |
| Foreign Patent | A-0627259 | EP | 1994 |
| Documents | | | |

It is known that the outer layers of human skin can be caused to peel by applying preparations to remove dead skin without wounding underlying living skin tissue. The beneficial result of such skin peeling is that when underlying layers of new skin are exposed, the new skin is relatively free of wrinkles, dryness, pigmentation, and aging spots, and the skin is more youthful looking. Removing old, dead, skin cells, to expose younger looking skin, can be accomplished by exfoliating, since exfoliation removes only dead skin cells from the skin surface and does not wound living cells.

Various attempts have been made to utilize hydroxyacids in skin care products, as disclosed in the cited prior art. However, it should be noted that none of the cited prior art discloses use of hydroxyacids, safflower oil, flaxseed oil, organic beeswax, and tincture of benzoin to facilitate peeling and exfoliation in a moist, hydrating manner, as this present invention does.

The cosmetic and pharmaceutical industry is continually expanding its efforts in order to provide topical preparations which will exfoliate and peel the skin, eliminate dryness, wrinkles, photoaging, and restore the skin to a smooth condition, thus the combinations of skin aggravating synthetic compositions employed continue to increase yearly.

Synthetic surfactants, silicones, ammonia, alcohol's, solvents, artificial colors, hydrogen peroxide, esters, kojic acid, hydrocarbons, fungicides, mineral oils, bicarbonates, sulfurs, zinc complexes, salicylic acid, petrolatum, retinoids, enzymes, gums, tannic acid, polymers, dyes, and in-organic fragrances, and the like are being incorporated into the compositions of the cited prior art that are supplied to salons, stores, and supermarkets.

It will be appreciated that the expansion and growth of the cited prior art has devolved into highly complex synthetic adjuvants for use in the formulations.

The problem with the use of the compositions of the cited prior art which make up the bulk of transdermal delivery systems are their chemical structures which are known to cause skin irritations and skin sensitivities.

Synthetic surfactants, silicones, ammonia, alcohol's, solvents, artificial colors, hydrogen peroxide, esters, kojic acid, hydrocarbons, fungicides, mineral oils, bicarbonates, sulfurs, zinc complexes, salicylic acid, petrolatum, retinoids, enzymes, gums, tannic acid, polymers, dyes, acetones, and inorganic fragrances, incorporated into the compositions of the prior art cause dryness of the skin, rashes, allergies, dermatitis, bleeding, itching, painful cracking, peeling, and flaking of the skin.

Unfortunately, the cited prior art compositions are relatively ineffective for allowing the treatment of conditions related to sensitive and allergic skin reactions to silicones, ammonia, alcohol's, solvents, artificial colors, hydrogen peroxide, esters, kojic acid, hydrocarbons, fungicides, mineral oils, bicarbonates, sulfurs, zinc complexes, petrolatum, retinoids, enzymes, gums, tannic acid, polymers, dyes, acetones, and inorganic fragrances, so there is a need in the art for organic compounds for improved and useful skin care compositions to exfoliate the skin, without substantial irritation.

The said prior art compositions do not provide for exfoliation of skin in a gentle manner as does the present invention.

The said prior art compositions do not employ the massive quantities of cold-pressed organic vegetable oils, and organic beeswax, as does the present invention.

The said prior art compositions do not provide a moist exfoliation that enables dead skin cells to be shed without visible flaking, drying, facial tautness, and shedding of the skin.

The said prior art compositions do not provide compositions that employ hydroxyacids with high volumes of organic polyunsaturated oil lipids in their formulations as does the present invention.

The said prior art compositions do not employ organic beeswax and hydroxyacids in their compositions as does the present invention.

The said prior art compositions do not employ tincture of benzoin, which is heated to remove its alcohol content as does the present invention.

The said prior art compositions do not provide satisfactory skin penetration of the acids of hydroxyacids due to the inability of mineral oil, silicon, and petrolatum based components to penetrate the dermis, thereby hindering immediate and sustained delivery of the hydroxyacids to the dermis.

In terms of effectiveness, the said cited prior art compositions do not provide satisfactory skin penetration and supplying properties to leave skin satiny smooth for up to twelve hours.

In terms of effectiveness, the said cited prior art compositions do not provide a composition that is resistant to body fluids, sebaceous activity and perspiration, as does the present invention.

The said cited prior art compositions do not provide the high volume of hydroxyacids mixed with the high volume of cold pressed organic safflower oil, flaxseed oil, and tocopheral linoleate in their formulations to restore essential oil lipids that are needed to sustain radiant, glowing skin during the employment of hyroxyacids.

The said cited prior art compositions do not provide the high volume of skin healing vitamin A and tocopheral linoleate compositions to their formulations due to the prohibitively expensive manufacturing costs.

The said cited prior art compositions do not boil tincture of benzoin to eliminate its alcohol content.

The said cited prior art compositions do not provide an urgent need that exists for compositions like the present invention to the dermatological, pharmaceutical, alternative therapy beauty, and cosmetology fields.

The said cited prior art compositions do not provide organic oil lipids and hydroxyacids compositions to alleviate and treat the symptoms of sensitive skin infirmities.

The said cited prior art compositions do not provide an organic, liquid composition containing hydroxyacids for use in treating and alleviating, dry, aged, and photoaged, skin conditions, as does the present invention.

The said cited prior art compositions do not provide organic moisturizing compositions that render a water resistant, non-greasy, antiseptic film on the skin during dermal exfoliation.

The said cited prior art compositions do not provide organic compositions that are resistant to body fluids, sebaceous gland activity and perspiration.

The said cited prior art compositions do not provide organic compositions to provide a high volume of organic essential oil of lavender for anti-inflammatory qualities and to perfume the said compositions.

The said cited prior art compositions do not provide a high volume of organic safflower and flaxseed oils in the said compositions.

The said cited prior art compositions do not provide high volume organic compositions to replace lost oil lipids during exfoliation to the dermis.

The said cited prior art compositions do not provide gentle, organic compositions for adolescent use.

Objectives and advantages of the present invention are:

a) to provide an organic exfoliating and peeling skin preparation,

b) to provide hyroxyacids, safflower oil, flaxseed oil, organic beeswax, and tincture of benzoin to facilitate peeling and exfoliation of the skin in a moist, hydrating manner;

c) to provide a topical skin composition that is free of surfactants, silicones, ammonia, alcohol, solvents, artificial colors, hydrogen peroxide, esters, kojic acid, hydrocarbons, fungicides, mineral oil, bicarbonates, sulfurs, zinc complexes, petrolatum, retinoids, enzymes, gums, tannic acid, polymers, dyes, and non organic fragrances;

d) to provide effective organic compounds to exfoliate the skin without substantial irritation,

e) to provide a potent cold-pressed organic vegetable oils, and organic beeswax in the exfoliating and peeling composition,

f) to provide a composition that enables dead skin cells to exfoliate without visible flaking, drying, peeling, facial tautness, and shedding of skin;

g) to provide organic beeswax and hydroxyacids in the composition,

h) to provide high volumes of polyunsaturated oil lipids in the composition,

i) to provide tincture of benzoin, which is heated to eliminate its alcohol content,

j) to provide an organic beeswax substance that bonds lubricating, hydrating,

k) to provide satisfactory skin penetration of the hydroxyacids without the use of mineral oil, silicon, and petrolatum,

l) to provide effective treatment of conditions related to sensitive and allergic skin reactions to silicones, ammonia, alcohol, solvents, artificial colors, hydrogen peroxide, esters, kojic acid, hydrocarbons, fungicides, bicarbonates, sulfurs, zinc, retinoids, enzymes, gums, tannic acids, polymers, dyes, acetones, and inorganic fragrances;

n) to provide an urgent need in art for a composition like the present invention,

o) to provide satisfactory skin penetration properties that leave the skin satiny smooth for up to twelve hours,

p) to provide a composition that is resistant to body fluids, sebaceous activity, and perspiration,

q) to provide the high volume of hydroxyacids, and organic oil lipids, that are essential to sustain radiant glowing skin during the exfoliating process;

r) to provide a high volume of skin healing vitamin A and tocopheral linoleate,

s) to provide tincture of benzoin as an antimicrobial,

t) to provide compositions like the present invention to the dermatological, pharmaceutical, alternative therapy, beauty, and cosmetology fields;

u) to provide organic oil lipids and hydroxyacids for symptoms of sensitive skin infirmities,

v) to provide hyroxyacids and organic oil lipids in an oily liquid composition for treating and alleviating, severely dry, aged, and photodamaged skin,

w) to provide a water resistant, antiseptic film on the skin during exfoliation,

x) to provide a high volume of organic oil for anti-inflammatory qualities and to perfume the present invention,

y) to provide organic composition to replace lost lipids from the skin during exfoliation; and

z) to provide gentle, organic exfoliating compositions for adolescent use.

An urgent need exists for an improved and useful skin composition like the present invention, that employs a high volume of hydroxyacids, and potent cold-pressed organic flaxseed and safflower oils, plus organic vitamins to nourish and exfoliate the skin without the addition of skin irritating synthetic adjuvants to achieve its purposes. Further advantages of this invention are the employment of an organic wax substance and an organic antiseptic substance that bonds to the skin to provide superior skin penetrating properties of the compositions constituents, and maintains the skin in glowing, radiant condition for up to twelve hours. Still further objects and advantages will become apparent from a consideration of the ensuing descriptions.

### BRIEF SUMMARY OF THE INVENTION

An improved and useful liquid skin treatment for the maintenance of youthful, glowing, and hydrated skin, including a sufficient amount of an hydroxyacid skin exfoliating agent, to facilitate exfoliation of skin in a novel moist manner, without substantial irritation, as well as the prevention, alleviation and management of skin conditions, such as wrinkled, irregularly pigmented, photoaged, juvenile and adult acne, and thickened layers of the epidermis, and the like, which has as its constituents, safflower oil, flaxseed oil, sweet almond oil, apricot kernel oil, jojoba oil, organic beeswax, stearic acid, cetearyl alcohol, cocoa butter, vitamin A, tocopheral linoleate, borax, a perfuming agrent such as essential oil of lavender, peppermint, orange, lemon, grapefruit, spearmint, rosemary, violet, sandalwood, musk, magnolia, citrus, vanilla, gardenia, papaya, peach, rose, verbena, lilac, rosewood, neroli, ginger, almond, and the like, and tincture of benzoin as an antimicrobial agent to inhibit microorganisms on the skin.

### DESCRIPTION OF THE INVENTION

A composition for the maintenance of youthful, glowing, and radiant skin, including a sufficient amount of organic oil lipids, lactic acid, citric acid, malic acid, willow bark and green tea extract, to facilitate exfoliation of skin in a novel moist manner. This dual action approach gives a deep and more effective exfoliation result while maintaining the radiant hydration of the skin.

It is an objective of the improved and useful invention to provide an organic exfoliating and peeling skin preparation.

It is an objective of the improved and useful invention to provide hyroxyacids, safflower oil, flaxseed oil, organic beeswax, and tincture of benzoin to facilitate peeling and exfoliation of the skin in a moist, hydrating manner.

It is an objective of the improved and useful invention to provide a topical skin composition that is free of surfactants, silicones, ammonia, alcohol, solvents, artificial colors, hydrogen peroxide, esters, kojic acid, hydrocarbons, fungicides, mineral oil, bicarbonates, sulfurs, zinc complexes, petrolatum, retinoids, enzymes, gums, tannic acid, polymers, dyes, and non organic fragrances.

It is an objective of the improved and useful invention to provide effective organic compounds to exfoliate the skin without substantial irritation.

It is an objective of the improved and useful invention to provide a potent cold-pressed organic vegetable oils, and organic beeswax in the exfoliating and peeling composition.

It is an objective of the improved and useful invention to provide a composition that enables dead skin cells to exfoliate without visible flaking, drying, peeling, facial tautness, and shedding of skin.

It is an objective of the improved and useful invention to provide organic beeswax and hydroxyacids in the composition.

It is an objective of the improved and useful invention to provide high volumes of polyunsaturated oil lipids in the composition.

It is an objective of the improved and useful invention to provide tincture of benzoin, which is heated to eliminate its alcohol content.

It is an objective of the improved and useful invention to provide an organic beeswax substance that bonds lubricating, hydrating.

It is an objective of the improved and useful invention to provide satisfactory skin penetration of the hydroxyacids without the use of mineral oil, silicon, and petrolatum.

It is an objective of the improved and useful invention to provide effective treatment of conditions related to sensitive and allergic skin reactions to silicones, ammonia, alcohol, solvents, artificial colors, hydrogen peroxide, esters, kojic acid, hydrocarbons, fungicides, bicarbonates, sulfurs, zinc, retinoids, enzymes, gums, tannic acids, polymers, dyes, acetones, and inorganic fragrances.

It is an objective of the improved and useful invention to provide an urgent need in the art for a composition like the present invention.

It is an objective of the improved and useful invention to provide satisfactory skin penetration properties that leave the skin satiny smooth for up to twelve hours.

It is an objective of the improved and useful invention to provide a composition that is resistant to body fluids, sebaceous activity, and perspiration.

It is an objective of the improved and useful invention to provide the high volume of hydroxyacids, and organic oil lipids, that are essential to sustain radiant glowing skin during the exfoliating process.

It is an objective of the improved and useful invention to provide a high volume of skin healing vitamin A and tocopheral linoleate.

It is an objective of the improved and useful invention to provide tincture of benzoin as an antimicrobial.

It is an objective of the improved and useful invention to provide compositions like the present invention to the dermatological, pharmaceutical, alternative therapy, beauty, and cosmetology fields.

It is an objective of the improved and useful invention to provide organic oil lipids and hydroxyacids for symptoms of sensitive skin infirmities.

It is an objective of the improved and useful invention to provide hyroxyacids and organic oil lipids in an oily composition for treating and alleviating, severely dry, aged, and photodamaged skin.

It is an objective of the improved and useful invention to provide a water resistant, antiseptic film on the skin during exfoliation.

It is an objective of the improved and useful invention to provide a high volume of organic oil for anti-inflammatory qualities and to perfume the present invention with an oil type chosen from a group comprising essential oil of lavender, peppermint, orange, lemon, grapefruit, spearmint, rosemary, violet, sandalwood, musk, magnolia, citrus, vanilla, gardenia, papaya, peach, rose, verbena, lilac, rosewood, neroli, ginger, almond, and the like.

It is an objective of the improved and useful invention to provide organic composition to replace lost lipids from the skin during exfoliation.

It is an objective of the improved and useful invention to provide gentle, organic exfoliating compositions for adolescent use.

These and other objects are accomplished in accordance with a particular embodiment of the present improved and useful invention which provides a skin care composition in the form of a cream, comprising a mixture consisting of lactic acid, citric acid, malic acid, willow bark and green tea extract, safflower oil, flaxseed oil, sweet almond oil, apricot kernel oil, jojoba oil, organic beeswax, stearic acid, cetearyl alcohol, cocoa butter, vitamin A and tocopheral linoleate, borax, a perfuming agent such as essential oil of lavender, and tincture of benzoin, and mixtures thereof. The essential components of the present improved composition readily intermix to form an improved mixture suitable for topical applications to the skin. The present components are combined in such relative proportions so as to bring about a marked beautifying effect to the skin by supplying to the skin nutrients that are essential for healthy, radiant, skin. The present improved invention includes the surprising discovery that cold-pressed organic safflower and flaxseed oils, tincture of benzoin, high volumes of vitamins A, tocopheral linoleate and hydroxyacids, can be used as active principals in the improved moist exfoliation skin care composition to treat conditions attributed to junior and adult acne, wrinkled, irregularly pigmented, photoaged, dry, and thickened layers of the epidermis conditions.

In accordance with the present improved invention there is provided a formulation where cold-pressed, organic safflower oil, flaxseed oil, and tincture of benzoin can be employed with organic beeswax and hydroxyacids to aid in the water-resistant treatment and alleviation of skin conditions. The embodiment of the improved invention follow to illustrate the formulation.

The main ingredients of a first embodiment of this lotion are:

900 milliliters water,

150 milliliters composition comprising 92% lactic acid, 2% citrus (lemon and orange) extract, and 2% apple extract, 2% willow bark extract, and 2% green tea extract,

60 grams cocoa butter,

30 milliliters cold-pressed safflower oil,

30 grams cetearyl alcohol,

16.8 grams sodium behenoyl lactylate,

15 grams glycol stearate,

30 milliliters sweet almond oil,

10 milliliters apricot kernel oil,

10 milliliters glycerin,

10 milliliters tocopheral linoleate,

5 milliliters composition containing 35,000 micrograms Vitamin A,

5 milliliters flaxseed oil,

5 milliliters perfuming ingredient such as essential oil of lavender,

5 milliliters tetra sodium EDTA,

4 grams carbomer,

1.25 milliliters dmdm hydantoin,

0.8 milliliters triethanolamine; and

0.6 milliliters tincture of benzoin.

The main ingredients of a second embodiment of this emulsion are:

15 grams organic beeswax,

15 grams cetearyl alcohol,

45 milliliters safflower oil,

45 milliliters sweet almond oil,

36 milliliters lactic acid,

30 milliliters water,

30 milliliters apricot kernel oil,

15 milliliters jojoba oil,

10 milliliters flaxseed oil,

5 milliliters tocopheral linoleate,

5 milliliters perfuming ingredient such as essential oil of lavender,

5 milliliters composition containing 30,000 micrograms vitamin A,

0.12 milliliters tetra sodium EDTA,

0.6 milliliters dmdm hydantoin,

0.4 milliliters triethanolamine; and

0.3 milliliters tincture of benzoin.

My lotion process is as follows:

adding in a container being heated while said container is partially submersed in a 80 degree C. bath, sodium behenoyl lactylate, safflower oil, sweet almond oil, apricot kernel oil, tocopheral linoleate, vitamin A, flaxseed oil, cetearyl alcohol, glycol stearate, cocoa butter and glycerin to form a mixture,

stirring said mixture until dissolved,

adding simultaneously in a second container, while said second container is partially submersed in an 80 degree C. bath, water, tetra sodium EDTA, carbomer, and tincture of benzoin,

stirring said second mixture until dissolved to allow alcohol content in tincture of benzoin to burn off,

removing said mixtures from heat and blending both mixtures until said mixtures cool to 40 degrees C.;

adding perfuming agent such as essential oil of lavender, and dmdm hydantoin, triethanolamine, and composition comprising lactic acid, citrus extract, apple extract, willow bark extract, green tea extract; and

blending said mixture until it thickens, whereby the ingredients are formed into a lotion.

It is believed that the moisturizing, exfoliating, softening, long term delivery properties of organic cold-pressed flaxseed oil, organic cold-pressed safflower oil, and the high volume of vitamins A and tocopheral linoleate, combined with lactic acid and tincture of benzoin, emulsified in organic beeswax are responsible for the moist exfoliation of the skin, but we don't wish to be bound by this.

As noted, the improved composition hereof comprises a water-resistant, topical application which is applied to the dermal areas of the body to alleviate, treat, and manage skin conditions, such as wrinkled, irregularly pigmented, aged, photoaged, and thickened layers of the epidermis, by feeding aged skin the essential polyunsaturated oil lipids it needs to maintain its healthy condition, whilst supplying to the skin, deeply penetrating oil lipids that act as swift delivery vehicles for acids of hydroxyacids to institute a moist, invisible, exfoliation of the skin cells.

This improved formulation is highly effective for cosmetic and pharmaceutical use in the treatment and management of skin conditions, such as wrinkled, irregularly pigmented, aged, photoaged, and thickened layers of the epidermis, and the like, due to the resistance of the formulation to attack by body liquids, sebaceous oils and perspiration, thereby permitting sustained advantageous skin absorption of the hyroxyacids.

This improved formulation provides a water-resistant, light-weight, invisible composition that is preferred to be used during the daytime.

This improved formulation employs organic polyunsaturated oil lipids in the place of mineral oil, which is known for its lack of skin penetrating properties, thereby limiting delivery of hydroxyacids deep into the skin cells.

This improved formulation provides compositions like the present invention to the dermatological, pharmaceutical, alternative therapy, beauty, and cosmetology fields.

This improved formulation provides a topical skin composition that is free of surfactants, silicones, ammonia, alcohol, solvents, artificial colors, hydrogen peroxide, esters, kojic acid, hydrocarbons, fungicides, mineral oil, bicarbonates, sulfurs, zinc complexes, petrolatum, retinoids, enzymes, gums, tannic acid, polymers, dyes, and non organic fragrances.

This improved formulation provides effective organic compounds and hydroxyacids to exfoliate the skin without substantial irritation.

This improved formulation provides potent cold-pressed organic vegetable oils, and organic beeswax in the exfoliating and peeling composition.

This improved formulation provides a composition that enables dead skin cells to exfoliate without visible flaking, drying, peeling, facial tautness, and shedding of skin.

This improved formulation provides organic beeswax and hydroxyacids in the composition.

This improved formulation provides high volumes of polyunsaturated oil lipids in the composition.

This improved formulation provides tincture of benzoin, which is heated to eliminate its alcohol content.

This improved formulation provides an organic beeswax substance that bonds lubricating, hydrating, exfoliating compounds to the skin, thereby allowing sustained skin absorption of said constituents.

This improved formulation provides satisfactory skin penetration of the hydroxyacids without the use of mineral oil, silicon, and petrolatum.

This improved formulation provides effective treatment of conditions related to sensitive and allergic skin reactions to silicones, ammonia, alcohol, solvents, artificial colors, hydrogen peroxide, esters, kojic acid, hydrocarbons, fungicides, bicarbonates, sulfurs, zinc, retinoids, enzymes, gums, tannic acids, polymers, dyes, acetones, and inorganic fragrances.

This improved formulation provides an urgent need in the art for a composition like the present invention.

This improved formulation provides satisfactory skin penetration properties that leave the skin satiny smooth for up to twelve hours.

This improved formulation provides a composition that is resistant to body fluids, sebaceous activity, and perspiration.

This improved formulation provides the high volume of hydroxyacids, and organic oil lipids, that are essential to sustain radiant glowing skin during the exfoliating process.

This improved formulation provides a high volume of skin healing vitamin A and tocopheral linoleate.

This improved formulation provides tincture of benzoin as an antimicrobial.

This improved formulation provides organic oil lipids and hydroxyacids for symptoms of sensitive skin infirmities.

This improved formulation provides hydroxyacids and organic oil lipids in an oily composition for treating and alleviating, severely dry, aged, and photodamaged skin.

This improved formulation provides a water resistant, antiseptic film on the skin during exfoliation.

This improved formulation provides a high volume of organic oil for anti-inflammatory qualities and to perfume the present invention.

This improved formulation provides organic compostion to replace lost lipids from the skin during exfoliation.

This improved formulation provides provide gentle, organic, exfoliating compositions for adolescent use.

For a more complete understanding of the present improved composition reference is made to the following examples. The following examples are illustrative of the present improved composition and are not intended in any as a limitation upon the scope thereof.

### EXAMPLE 1

To evidence the efficacy of the improved lotion and emulsion, a series of individuals suffering from various skin infirmities were treated with the improved formulation. Compared to commercially available skin exfoliants, and acne products, the results based upon the efficacy of the present invention are set forth hereinafter:

a) After three days use, a 13 year old female user found her facial skin to have a dramatic reduction of a persistant acne condition, her skin was softer and very smooth, with visible reduction of the acne. User found present lotion invention superior to any commercially available acne products.

b) A 17 years old female user found the present invention eliminated the acne lesions from the skin of her face and chest after one application of present lotion invention, and the skin was soft, smooth, and without dryness or redness.

d) A 50 year old female user suffering from dry, photoaged skin, found the emulsion rehydrated her skin within two days use. The skin became pliable, soft and glowing withing 3 days use. Ater ten days use, the upper layers of the dermis had exfoliated in an invisible moist manner, and new radiant skin was visible over the entire facial area.

e) An 18 year old female suffering from severe acne, applied lotion composition to the skin of her face. After one nightly application, the acne on the skin was greatly reduced, the skin was soft, and displayed no redness. The said user, had been on retinol treatment for the past three years, with only mild success in controlling her acne. User found present lotion invention superior to any commercial treatment she had undertaken to control and treat her acne.

f) A 48 year old male, found the emulsion superior for exfoliating dry, wrinkled skin from his forehead. Twice daily applications were used for five days before soft, smooth skin was visible.

Thus, from the preceding it will be readily perceived by the reader that an urgent need exists for this improved composition for the alleviation of skin conditions denoted heretofore, but which also, can be used to prevent such conditions from occurring;

The improved composition provides:

a water-resistant, topical application to alleviate, treat, and manage skin conditions, such as wrinkled, irregularly pigmented, aged, photoaged, and thickened layers of the epidermis, by feeding aged skin the essential polyunsaturated oil lipids it needs to maintain its healthy condition, whilst supplying to the skin, deeply penetrating oil lipids that act as swift delivery vehicles for acids of hydroxyacids to institute a moist, invisible, exfoliation of the skin cells,

an improved formulation that is highly effective for cosmetic and pharmaceutical use in the treatment and management of skin conditions, such as wrinkled, irregularly pigmented, aged, photoaged, and thickened layers of the epidermis, and the like, due to the resistance of the formulation to attack by body liquids, sebaceous oils and perspiration, thereby permitting sustained advantageous skin absorption of the hydroxyacids,

an improved formulation that provides a light-weight, invisible composition that is preferred to be used during the daytime,

an improved formulation that employs organic polyunsaturated oil lipids in the place of mineral oil, and petrolatum,

a need that exists for the improved formulation like the present invention to the dermatological, pharmaceutical, alternative therapy, beauty, and cosmetology fields,

a topical skin composition that is free of surfactants, ammonia, alcohol, solvents, artificial colors, hydrogen peroxide, esters, kojic acid, hydrocarbons, fungicides, bicarbonates, sulfurs, zinc complexes, petrolatum, retinoids, enzymes, gums, tannic acid, polymers, dyes, and non organic fragrances,

effective organic compounds and hydroxyacids to exfoliate the skin without substantial irritation,

potent cold-pressed organic vegetable oils, and organic beeswax in the exfoliating composition,

a composition that enables dead skin cells to exfoliate without visible flaking, drying, peeling, facial tautness, and shedding of skin,

organic beeswax and hydroxyacids in the composition,

high volumes of cold-pressed polyunsaturated oil lipids in the composition,

tincture of benzoin, which is heated to eliminate its alcohol content,

organic beeswax to bond lubricating, hydrating, exfoliating compounds to the skin, thereby allowing sustained and deep skin absorption of said constituents,

satisfactory skin penetration of the hydroxyacids without the use of mineral oil, silicon, and petrolatum,

effective exfoliation treatment of conditions related to sensitive and allergic skin reactions to silicones, ammonia, alcohol, solvents, artificial colors, hydrogen peroxide, esters, kojic acid, hydrocarbons, fungicides, bicarbonates, sulfurs, zinc, retinoids, enzymes, gums, tannic acids, polymers, dyes, acetones, and inorganic fragrances,

satisfactory skin penetration properties that leave the skin satiny smooth for up to twelve hours,

a composition that is resistant to sebaceous activity, and perspiration,

a high volume of hydroxyacids, and organic oil lipids, that are essential to sustain radiant glowing skin during the exfoliating process,

a high volume of skin healing vitamin A and tocopheral linoleate,

tincture of benzoin as an antimicrobial,

organic oil lipids and hydroxyacids for symptoms of sensitive skin infirmities,

hydroxyacids and organic oil lipids in an oily composition for treating and alleviating, severely dry, aged, and photodamaged skin,

a water resistant, antiseptic film on the skin during exfoliation,

a high volume of organic oil for anti-inflammatory qualities and to perfume the present invention,

an organic composition to replace lost lipids from the skin during exfoliation; and

a gentle, organic, exfoliating compositions suitable for adolescent use.

While the above description contains many specificity's, these should not be construed as limitations on the scope of the improved invention, but merely providing illustrations of some of the presently preferred embodiments of this improved invention.

Many other variations are possible. For example, it is additionally possible to add to the present invention many other adjuvants such as ultraviolet filters, color pigments, pearling pigments, hydrating agents, additional essential plant oils and perfuming ingredients, vitamins, antibiotics, anti-inflamatory agents, additional plant and vegetable oils and be within the purview of this invention.

Accordingly, the scope of the invention should be determined by the specification and the appended claims and their legal equivalents, rather than only by the examples given.

What is claimed is:

1. A lotion comprising:
from 50 to 2000 milliliters water,
from 10 to 800 milliliters composition comprising 92% lactic acid, 2% citrus (lemon and orange) extract, and 2% apple extract, 2% willow bark extract, and 2% green tea extract,
from 2 to 300 grams cocoa butter,
from 0.1 to 500 milliliters cold-pressed safflower oil,
from 5 to 400 grams cetearyl alcohol,
from 1 to 300 grams sodium behenoyl lactylate,
from 1 to 300 grams glycol stearate,
from 1 to 500 milliliters sweet almond oil,
from 1 to 500 milliliters apricot kernel oil,
from 1 to 200 milliliters glycerin,
from 0.001 to 200 milliliters tocopheral linoleate,
from 0.001 to 200 milliliters composition containing 35,000 micrograms Vitamin A,
from 0.001 to 400 milliliters flaxseed oil,
from 0.1 to 200 milliliters of a perfuming agent chosen from a group comprising oil of lavender, peppermint, orange, lemon, grapefruit, spearmint, rosemary, violet, sandalwood, musk, magnolia, citrus, vanilla, gardenia, papaya, peach, rose, verbena, lilac, rosewood, neroli, ginger, almond, geranium, cinnamon, nutmeg, lime, sage, rosewood, tea tree, eucalyptus, thyme, ylang ylang,
from 0.001 to 100 milliliters tetra sodium EDTA,
from 1 to 200 grams carbomer,
from 0.001 to 60 milliliters dmdm hydantoin,
from 0.001 to 100 milliliters triethanolamine; and
from 0.001 to 200 milliliters tincture of benzoin.

2. A lotion comprising:
900 milliliters water,
150 milliliters composition comprising 92% lactic acid, 2% citrus (lemon and orange) extract, and 2% apple extract, 2% willow bark extract, and 2% green tea extract,
60 grams cocoa butter,
30 milliliters cold-pressed safflower oil,
30 grams cetearyl alcohol,
16.8 grams sodium behenoyl lactylate,
15 grams glycol stearate,
30 milliliters sweet almond oil,
10 milliliters apricot kernel oil,
10 milliliters glycerin,
10 milliliters tocopheral linoleate,
5 milliliters composition containing 35,000 micrograms Vitamin A,
5 milliliters flaxseed oil,
5 milliliters perfuming ingredient such as essential oil of lavender,
5 milliliters tetra sodium EDTA,
4 grams carbomer acritamer,
1.25 milliliters dmdm hydantoin,
0.8 milliliters triethanolamine; and
0.6 milliliters tincture of benzoin.

3. An emulsion comprising:
from 1 to 100 grams organic beeswax,
from 1 to 200 grams cetearyl alcohol,
from 0.1 to 300 milliliters safflower oil,
from 0.1 to 300 milliliters apricot kernel oil,
from 0.1 to 300 milliters sweet almond oil,
from 0.1 to 400 milliliters jojoba oil,
from 0.01 to 300 milliliters flaxseed oil,
from 0.01 to 100 milliliters tocopheral linoleate,
from 0.01 to 80 milliliters vitamin A,
from 1 to 500 milliliters water,
from 0.1 to 300 milliliters of an hydroxyacid skin exfoliation means,
from 0.01 to 50 milliliters tetra sodium EDTA,
from 0.01 to 80 milliliters dmdm hydantoin,
from 0.1 to 100 milliliters of a perfuming agent chosen from a group comprising oil of lavender, peppermint, orange, lemon, grapefruit, spearmint, rosemary, violet, sandalwood, musk, magnolia, citrus, vanilla, gardenia, papaya, peach, rose, verbena, lilac, rosewood, neroli, ginger, almond, geranium, cinnamon, nutmeg, lime, sage, rosewood, tea tree, eucalyptus, thyme, ylang ylang,
from 0.001 to 20 milliliters triethanolamine, and
from 0.01 to 100 milliliters tincture of benzoin.

4. An emulsion comprising:
15 grams organic beeswax,
15 grams cetearyl alcohol,
45 milliliters safflower oil,
45 milliliters sweet almond oil,
36 milliliters lactic acid,
30 milliliters water,
30 milliliters apricot kernel oil,
15 milliliters jojoba oil,
10 milliliters flaxseed oil,
5 milliliters tocopheral linoleate,
5 milliliters of an perfuming agent such as essential oil of lavender,
5 milliliters composition containing 30,000 micrograms vitamin A,
0.12 milliliters tetra sodium EDTA,
0.6 milliliters dmdm hydantoin,
0.4 milliliters triethanolamine; and
0.3 milliliters tincture of benzoin.

5. A method of making a lotion comprising:
adding in a container being heated while said container is partially submersed in a 100 degree C. bath, sodium behenohl lactylate, safflower oil, sweet almond oil, apricot kernel oil, tocopheral linoleate, vitamin A, flaxseed oil, cetearyl alcohol, glycol stearate, cocoa butter and glycerin to form a mixture,
stirring said mixture until dissolved;
adding simultaneously in second container while said second container is being heated while partially submersed in a 100 degree C. bath, water, tetra sodium EDTA, carbomer, and tincture of benzoin,
stirring said second mixture until mixture reaches 80 degrees C., to boil off alchohol content of tincture of benzoin,
removing said mixtures from heat and blending both mixtures until said mixtures cool to 40 degrees C.;
adding slowly while stirring, a perfuming agent such as essential oil of lavender, dmdm hydantoin, triethanolamine, and composition comprising hydroxyacids; and
blending said mixture until it thickens, whereby the ingredients are formed into a lotion.

6. A method of making an emulsion comprising:
adding in a container being heated while said container is partially submersed in a 100 degree C. bath, safflower oil, sweet almond oil, apricot kernel oil, tocopheral linoleate, vitamin A, jojoba oil, flaxseed oil, cetearyl alcohol, and beeswax, to form a mixture,
stirring said mixture until dissolved;
adding simultaneously in second container while said second container is being heated while partially submersed in a 100 degree C. bath, water, lactic acid, tetra sodium EDTA, and tincture of benzoin,
stirring said second mixture until mixture reaches 80 degrees C., to boil off alcohol content in tinture of benzoin,
stirring said mixture until dissolved;
removing said mixtures from heat and stirring said mixtures until said mixtures cool to 40 degrees C.;
adding of a perfiuming agent such as essential oil of lavender, and dmdm hydantoin, triethanolamine, and
blending said mixture until it thickens, whereby the ingredients are formed into an emulsion.
